(19) United States
(12) Patent Application Publication
(10) Pub. No.: US 2003/0073683 A1
(43) Pub. Date: Apr. 17, 2003

(54) Administration of noncompetitive AMPA receptor antagonist for neuroprotection after traumatic brain injury

(76) Inventors: Ludmila Belayev, Miami, FL (US); Myron D. Ginsburg, Coconut Grove, FL (US)

Correspondence Address: IVAX CORPORATION, 4400 Biscayne Boulevard, Miami, FL, 33137, US

(21) Appl. No.: 10/245,980

(22) Filed: Sep. 18, 2002

Related U.S. Application Data
(60) Provisional application No. 60/323,012, filed on Sep. 18, 2001.

(51) Int. Cl. A61K 31/551; A61K 31/724

(52) U.S. Cl. 514/220; 514/58

(57) ABSTRACT

The invention provides compositions and methods for treating traumatic brain injury by the administration of a noncompetitive AMPA receptor antagonist. In certain embodiments, the noncompetitive AMPA receptor antagonist is a 2,3-benzodiazepine derivative. Illustrative 2,3-benzodiazepine derivatives useful according to the invention include (R)-7-acetyl-5(4-aminophenyl)-8,9-dihydro-8-methyl-7H-1,3-dioxolo[4,5-h][2,3]benzodiazepine.

### CROSS-REFERENCE TO RELATED APPLICATION

This application claims priority to United States Provisional Patent Application Serial No. 60/323,012, filed on Sep. 18, 2001.

### TECHNICAL FIELD OF THE INVENTION

The present invention relates to the field α-amino-3-hydroxy-5-methyl-4-isoxazoic propionic acid (AMPA) receptor agonists.

### BACKGROUND OF THE INVENTION

Traumatic brain injury (“TBI”) represents one of the most tragic, common, and costly of all neurological disorders. Traumatic brain injury is associated with a large number of physical or behavioral disabilities. Each year in the United States, nearly 2 million people suffer from head injuries of sufficient severity to result in brain trauma (see Thurman et. al., J. Head Trauma Rehabil. 14: 602-615 (1999)).

Traumatic brain injury presents as a cascading continuum of symptomology ranging from concussion (a trauma induced alteration in the alert state) up to and including death, depending on the severity of the trauma inducing event. The initiating step for TBI will depend on the severity of the contusion (bruising) the trauma has produced in the brain and associated hemorrhage, if any, which may occur. The severity of the contusion and/or hemorrhage (and/or associated blood clots) may result in edema which, if severe, may occlude blood vessels producing cerebral ischemia or hypoxia. Cerebral ischemia and hypoxia if prolonged, can induce cell death. Depending on the location of the injury within the brain, the effects of the neuronal cell death may trigger such events as seizures, amnesia, or loss or impairment of autonomic functions resulting, at times, in death of the organism. A severe TBI can bypass the cascade sequence and directly cause death. While various treatment modalities have been investigated for ameliorating the damages and symptomology induced subsequent to the contusion event, little has been done to reduce or prevent the initial contusion, thereby minimizing or eliminating the damaging cascade itself.

Traumatic brain injury leads to early and marked increases in the excitatory amino acids glutamate and aspartate, with peak levels noted between 10 and 20 minutes (min) after trauma (see Globus et al., J. Neurochem., 65: 1704-1711 (1995); Nilsson et al., J. Cereb. Blood Flow Metab., 10: 631-637 (1990)). Antagonists of both the N-methyl-D-aspartate (NMDA) receptor/channel complex and the α-amino-3-hydroxy-5-methyl-4-isoxazoic propionic acid (AMPA) receptor may prevent injury-induced neuronal Ca²⁺ entry through these glutamate-regulated ionophores. As a result of both AMPA and NMDA receptor antagonists have been proposed as neuronal cytoprotectants against traumatic head injury (see McIntosh et al., Neuropathol. Appl. Neurobiol., 24: 251-267 (1998)). However, administration of both competitive (at the level of the receptor) and noncompetitive NMDA receptor antagonists (binding to a site within the opened NMDA receptor ion channel) have been tested for the treatment of traumatic injury and have been disappointing. Unfortunately, the few large clinical studies that have been initiated with glutamate antagonists have proven somewhat disappointing as well, and several have been terminated because of lack of efficacy or psychomimetic side effects (see Doppenberg et al., Neurotrauma, 14: 71-80 (1997)).

The 2,3-benzodiazepine class of compounds has been reported to possess anticonvulsive, muscle relaxant and neuroprotective activity for the central nervous system. For example, U.S. Pat. No. 4,835,152 postulates the central nervous system activity observed may be due to the influence of the benzodiazepines on dopaminergic mechanisms resulting in the modification of several functions of the nervous system (coordinated muscle contraction, learning processes, regulation of the blood pressure and the endocrine system). However, this work addresses events that are “downstream” to the initiating contusion.

U.S. Pat. No. 5,795,886 to Anderson, et al., at col. 8, lines 51-57, discloses a number of dihydro 2,3-benzodiazepine derivatives possessing anticonvulsant activity as measured by the method of Leander (Epilepsia., 33: 573-576 (1992)). However, the Leander test employed entails pretreatment prior to induction of electroshock seizure. This is distinguishable from the instant invention set forth hereinafter where the benzodiazepine treatment is initiated subsequent to the trauma event.

U.S. Pat Nos. 5,459,137 and 5,639,751 to Andrasi et al., describe several N-acyl-2,3-benzodiazepine derivatives with varying degrees of central nervous system activity, e.g., neuroprotective, anticonvulsant, and muscle relaxant activities. In these instances, treatment was directed towards amelioration of the symptomology associated with already established disease processes (e.g., epilepsy) or for prophylactic use (e.g., surgery-associated ischemic damage). By way of contrast, the instant invention addresses neural damage associated with TBI, i.e., to reduce or prevent subsequent development of neural damage which would otherwise materialize as a result of the processes triggered by the traumatic event itself.

The temporal relationship between TBI and drug treatment may be exemplified by experiments conducted with the selective, noncompetitive AMPA receptor antagonist GYKI 52466 (4-(8-methyl-9H-1,3dioxolo[4,5-h][2,3]benzodiazepin-5-yl)) on global cerebral ischemia in rats. See Block et al., J. Neurol. Sci., 139: 167-172, 1996. Drug pretreatment but not posttreatment reduced functional deficits (learned maze responses) and significantly attenuated neuronal damage in the CA1 sector of the hippocampus. Striatal neuronal damage was not affected by either drug treatment.

U.S. Pat. Nos. 4,983,586 & 5,024,998 disclose β-cyclodextrin formulations (20 to 50%) for numerous drugs including benzodiazepines. The cyclodextrins ability for form inclusion complexes with poorly soluble drugs has been further utilized in U.S. Pat. No. 5,017,566 as a means to specifically target drug delivery to the brain.

Cyclodextrins represent a group of cyclic oligomers of alpha-D-glucopyranose. The most common cyclodextrins are alpha, beta and gamma cyclodextrin having 6, 7 and 8 glucopyranose units respectively. Cyclodextrins, with their hydrophobic interior cavity can complex small hydrophobic molecules while remaining soluble in water which makes their use as carrier molecules for many pharmaceutical agents attractive.

Janssen Pharmaceutica N. V. in WO 85/02767 discloses oral, topical, and parenteral drug formulations (including derivatives of benzodiazepine but not talampanel) prepared in 4 to 10% hydroxyethyl- or 7 to 10% hydroxypropyl-β-cyclodextrins. To date, the United States Food and Drug Administration has approved formulations containing hydroxypropyl-β-cyclodextrins although various other approvals are pending including Pharmacia and Upjohn have proposed the use of α-cyclodextrin in their alprostadil product (AVERJECT®) and Janssen has proposed α-cyclodextrin use for their itraconazole product SPORANOX®.

Thus, there remains a need for effective therapeutic intervention for brain injury. Moreover, there is a need to further develop promising compounds for use as therapeutic agent for early onset traumatic brain injury.

### SUMMARY OF THE INVENTION

As discussed and shown in representative examples provided herein, it has been discovered that the administration of a noncompetitive antagonist of the AMPA subtype of glutamate excitatory amino acid receptors is therapeutically useful in the treatment of brain injury as evidenced by the reduction of the contusion areas and by the increased survival of pyramidal neurons of the CA1 sector of the hippocampus of treated animals.

Thus, the invention provides compositions and methods for treating brain injury (e.g., TBI) by the administration of a noncompetitive AMPA antagonist. In certain embodiments, the noncompetitive AMPA antagonist is a 2,3-benzodiazepine derivative. Illustrative 2,3-benzodiazepine derivatives useful according to the invention include (R)-7-acetyl-5(4-aminophenyl)-8,9-dihydro-8-methyl-7H-1,3-dioxolol[4,5-h][2,3]benzodiazepine also designated as talampanel.

These and other features of the invention will be further described and exemplified in the detailed description below.

### BRIEF DESCRIPTION OF THE DRAWINGS

FIG. 1 is a representation of the chemical structure of talampanel ((R)-7-Acetyl-5-(4-aminophenyl)-8, 9-dihydro-8-methyl-7H-1, 3-dioxolo [4, 5-h][2,3]benzodiazepine).

FIG. 2 is a representative set of photomicrographs of paraffin-embedded brain sections stained with hematoxylin and eosin 7 days following fluid-percussion brain injury in vehicle-treated and talampanel-treated rats. The photomicrographs are shown at two coronal levals (treatment was given at 30 min after trauma).

FIG. 3 is a set of bar graphs. (A) Contusion areas at six coronal levels in vehicle- and talampanel-treated rats at 7 days after trauma. Treatment was given 30 min after trauma (*p<0.05, Student's t test). (B) Total contusion area in vehicle- and talampanel-treated rats 7 days after trauma. Treatment was given at 30 min. after trauma (*p<0.05, Student's t test).

### DETAILED DESCRIPTION OF THE INVENTION

The methods and compositions disclosed herein capitalize on the discovery that AMPA antagonist therapy provides marked and significant reduction in neural damage following traumatic injury when administered within a short time proximate to the traumatic event. The reduction in neural damage observed is evidenced by diminution of the size of the cortical contusion area and increased survival of pyramidal neurons of the CA1 sector of the hippocampus.

The patent and scientific literature referred to herein establish the knowledge of those with skill in art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference is made in the following description and examples are obtainable from commercial sources, unless otherwise noted. In the specification and the appended claims, the singular forms include plural referents unless the context clearly dictates otherwise.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Thus, the invention provides methods for treating traumatic brain injury by the administration to a subject in need thereof of a therapeutically effective amount of a noncompetitive AMPA receptor antagonist within a therapeutically effective time window. In some embodiments, the noncompetitive AMPA receptor antagonist is a 2,3-benzodiazepine derivative. A 2,3-benzodiazepine derivative contemplated by the inventors and exemplified specifically to illustrate the invention is talampanel.

The methods of the present invention are intended for use with any subject that may experience the benefits of the methods of the invention. Thus, in accordance with the invention, “subjects” include humans as well as non-human subject, particularly domesticated animals.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration. The term “treating” also means protection, when the AMPA receptor antagonist is administered to the subject prior to the traumatic brain injury.

In animal models, glutamate is released relatively early after trauma, and most effective clinical treatment requires early intervention (see McIntosh, Cerebrovase. Brain Metab. Rev. 6: 109-162 (1994)). In Example 1, with moderate fluid-percussion brain injury, early post-treatment with talampanel given at 30 min after trauma led to significant reduction in overall contusion volume as well as increased survival of pyramidal neurons in the CA1 sector of hippocampus. By contrast, when talampanel treatment was begun at 3 hr, the neuroprotective effective of the drug was lost.

The term “effective amount” or “therapeutically effective amount” is usually an amount of AMPA antagonist that is capable of protecting from the effects of traumatic brain injury. Otherwise, the term “effective amount” is an amount of AMPA antagonist that is capable of blocking the AMPA excitatory amino acid receptor.

A proposed dose of the active compounds for use in the method of the invention for administration to the average adult human requiring treatment is 0.01 to 100 mg/kg of the active ingredient per unit dose which could be administered, for example, 1 to 4 times per day (i.e., divided dosing). More particularly, the dose can be 2.5-5 mg talampanel/kg body weight of subject; or about 4 mg talampanel/kg body weight of subject/hr.

The active compounds according to the invention may be formulated with a cyclodextrin carrier vehicle (e.g, 2-hydroxylpropyl-β-cylcodextrin) well known in the art (see e.g., U.S. Pat. Nos. 4,983,586 & 5,024,998 disclose β-cyclodextrin formulations (20 to 50%) for numerous drugs including benzodiazepines). The cyclodextrins ability for form inclusion complexes with poorly soluble drugs has been further utilized in U.S. Pat. No. 5,017,566 as a means to specifically target drug delivery to the brain.

As used herein, “therapeutically effective time window” means the time interval wherein administration of the compounds of the invention to the subject in need thereof reduces or eliminates the deleterious effects induced by brain injury. In a preferred embodiment, the compound of the invention is administered proximate to the event causing injury to the brain tissue. Hence, in some embodiments administration of the compound of the invention is initiated less than 3 hours following the event causing the injury. In other embodiments, administration is initiated less than 2.5 hours following injury. In another embodiment, administration is initiated less than 2 hours following injury. In yet another embodiment, the compound administration is initiated less than 1.5 hours following injury. In yet another embodiment, the compound administration is initiated less than 1 hour following injury. In yet another embodiment, the administration is initiated less than 0.5 hours following injury.

As used herein, “2,3 benzodiazepine” shall refer to the compounds composed of a benzene ring fused to a seven-membered diazepine ring, more specifically to several N-acyl-2,3-benzodiazepine derivatives with varying substituents attached as described in U.S. Pat Nos. 5,459,137 and 5,639,751, incorporated by reference in their entirety.

As used herein, “AMPA antagonist” means a compound capable of interfering with activation of the α-amino-3-hydroxy-5-methyl-4-isoxazolepropionate receptor. AMPA antagonists and method of making the same are well known in the art and are described in the scientific literature. Such compounds may be competitive or noncompetitive inhibitors of receptor activation. Representative “noncompetitive AMPA antagonists” include, without limitation, GYKI 52466 (4-(8-methyl-9H-1,3dioxolo[4,5-h][2,3]benzodiazepin-5-yl)), GYKI 53773 (talampanel, ((R)-7-Acetyl-5-(4-aminophenyl)-8, 9-dihydro-8-methyl-7H-1, 3-dioxolo [4, 5-h][2,3]benzodiazepine)), EGIS 8332, GYKI 47261, BIIR 561 CL, Phthalazinone, SYM 2207, 2,3-benzodiazepine-4-one, SYM 2189, or GYKI 54267 to mention a few.

Excitatory amino acids, mainly glutamate, are known to be the important excitatory neurotransmitter substances. Glutamate has an essential role in normal physiological processes in the central nervous system of mammals. Excitatory amino acids activate both ionotropic (ligand-gated ionic channels) and metabotropic (G-protein coupled) receptors, using second messenger systems. According to their sensitivities to different agonists, the ionotropic receptors were originally classified as NMDA, AMPA, and kainate (formerly quisqualate) receptors. Molecular biological studies confirmed this classification based on pharmacological sensitivities and revealed that several NMDA, AMPA, and kainate receptor subtypes exist. Several different AMPA receptor subunits are known. The terms AMPA/kainate receptors or non-NMDA receptors are sometimes used instead of AMPA receptors.

AMPA receptor antagonists of a competitive or noncompetitive nature can achieve inhibition of the activation of AMPA receptors. Noncompetitive antagonists, in general, offer advantages over competitive antagonists, as they provide better protection in situations when the extracellular concentration of excitatory amino acids is extremely high.

Beside several competitive AMPA receptor antagonists, two classes of noncompetitive antagonists are well known, the 2,3 benzodiazepines (see, e.g., U.S. Pat. No. 5,519,019 and U.S. Pat. No. 5,521,174) and the dye Evan's Blue. One highly selective antagonist noncompetitive AMPA-antagonist compound is 1-(4-aminophenyl)-4-methyl-7,8-methylene-dioxy-5H-2,3-benzodiazepine (GYKI 52466; U.S. Pat. No. 4,614,740). A new class of noncompetitive AMPA receptor antagonist has been derived from piraquilone and is typified by CP-465,022 and CP-453,850 (see, Menniti et al,. Mol. Pharmacol., 58:1310-1317 (2000)).

Numerous AMPA receptor antagonists (both competitive and noncompetitive) are described in several issued patents including: U.S. Pat. Nos. 5,654,303; 5,639,751; 5,614,532; 5,614,508; 5,606,062; 5,580,877; 5,559,125; 5,559,106; 5,532,236; 5,527,810; 5,521,174; 5,519,019; 5,514,680; 5,631,373; 5,622,952; 5,620,979; 5,510,338; 5,504,085; 5,475,008; 5,446,051; 5,426,106; 5,420,155; 5,407,935; 5,399,696; 5,395,827; 5,376,748; 5,364,876; 5,356,902; 5,342,946; 5,268,378; and 5,252,584. AMPA receptor antagonists (both competitive and noncompetitive) are also described in PCT international patent application WO 97/19066 as the compounds “NS-1201” or “NS-479”, developed or marketed by Neurosearch (Denmark); or as the compounds “LY-311446” (2-amino-3-(2-(3-(1H-tetrazol-5-yl)phenoxy)phenyl)propionic acid), “LY-300164” (7-acetyl-5-(4-aminophenyl)-8(R)-methyl-8,9-dihydro-7H-1,2-dioxolo(4,5-H)(2,3) benzodiazepine), “LY-293606”, “LY-293558”, or “GYKI-53655” of Eli Lilly (United States) in 20th CINP (Melbourne), Abs. S-40-1 (1996); the compound “NNC-07-0775” of Novo Nordisk (Denmark) in PCT international patent application WO 96/15100; the compound “SYM-2206” (4-(aminophenyl)-1-methyl-6,7-(methylenedioxy)-N-butyl-1,2-dihydrophthalazine-2-carboxamide) of Symphony Pharmaceuticals (United States) or any AMPA antagonist referred to in J. Med. Chem. 39: 343 (1996); the compound “A-17625” (6,7-dichloro-2(1H)-oxoquinoline-3-phosphonic acid) of Servier (France) or any AMPA antagonist referred to in J. Med. Chem. 39: 197 (1996);

2-carboxy-1-methyl-7-trifluoromethylimidazo(1,2-α)quinoxalin-4(5H)-one or any AMPA antagonist referred to in PCT international publication numbers WO 95/21842, WO 96/08492, and WO 96/08493; 6-(4-pyridinyl)-1H-1,2,3-triazolo(4,5-α)pyrimidin-4(5H)-one or any AMPA antagonist referred to in J. Med. Chem. 38: 587 (1995); any AMPA antagonist referred to in PCT international patent applications WO 94/26747, WO 95/19346, WO 95/12594, WO 95/02601, WO 95/26342, WO 95/26349, WO 95/26350, WO 95/26351, WO 95/26352, WO 96/31511, and WO 95/02602;

2-amino-3-(3-hydroxy-5-(2-thienyl)isoxazol-4-yl)propionic acid or any AMPA antagonist referred to in PCT international patent application WO 95/12587; the compound “SYM-2250” of Symphony Pharmaceuticals (United States); the compound “S-18986” of Servier (France) or any AMPA antagonist referred to in 13th Int. Symp. Med. Chem. (Paris), Abs. P29 (1994); the compound “NNC-07-9202 of Warner-Lambert (United States) or any AMPA antagonist referred to in 208th ACS (Washington, D.C.), Abs. MEDI 170 (1994); the compound ¢IDRA-21”

(7-chloro-3-methyl-3,4-dihydro-2H-1,2,4-benzothiadiazine-5,5-dioxide) or any AMPA antagonist referred to in Soc. Neurosci. Abstr., Abs. 124.7 and 124.8 (1993); the compound “NS-409” of Warner-Lambert (United States) or any AMPA antagonist referred to in J. Med. Chem. 38: 3720 (1995) or PCT international patent applications WO 96/08494 and WO 96/08495; the compound “NS-393” of Neurosearch (Denmark); the compounds “SYM-2101”, “SYM-2007” and “SYM-2057” of Symphony Pharmaceuticals (United States); the compound “AMPAlex” (1-(1,3-benzodioxolo-5-ylcarbonyl)piperidine) of Cortex Pharmaceuticals (United States) or any AMPA antagonist referred to in Scrip 2088/9 (1995), 14 and Scrip 2187, 21 (1996) or in PCT international patent application WO 96/38414; the compounds “LY-293558”, “LY-215490”, and decahydro-6-(2-(1H-tetrazol-5-yl)ethyl)-3-isoquinolinecarboxylic acid (CAS registry no. 154652-83-2) or any AMPA antagonist referred to in J. Med. Chem. 36: 2046 (1993); the compound “YM-90K”

(1,4-dihydro-6-(1H-imidazol-1-yl)-7-nitro-2,3-quinoxalinedione monohydrochloride (CAS registry no. 154164-30-4 or any AMPA antagonist referred to in Scrip 1972, 14 (1994) or PCT international patent application WO 96/10023; the compound “aloracetam” (N-(2-(3-formyl-2,5-dimethyl-1H-pyrrol-1-yl)ethyl)-acetamide) (CAS registry no. 119610-26-3) or any AMPA antagonist referred to in European Patent 0 287 988; the compound “NS-257” of Warner-Lambert; the compound “NNC-07-9202 of Novo Nordisk (Denmark) or any AMPA antagonist referred to in European Patent 0 283 959 and Science 241: 701 (1988); and the compound “aniracetam” of Roche (Switzerland) or 1-(4-methoxybenzyl)-2-pyrrolidinone (CAS registry no. 72432-10-1). Other AMPA receptor antagonists (both competitive and noncompetitive) are also known.

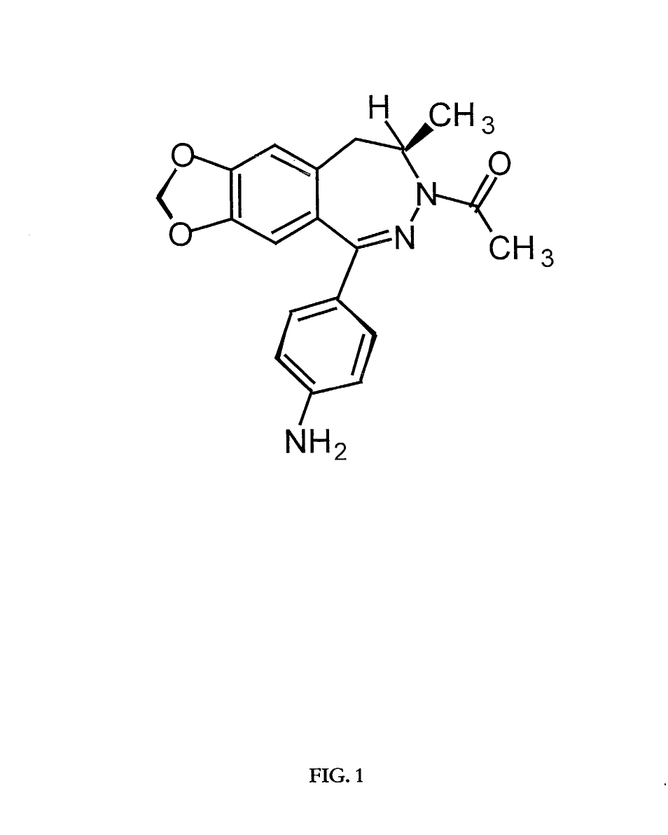

Talampanel [(R)-7-acetyl-5-(4-aminophenyl)-8, 9-dihydro-8-methyl-7H-1, 3-dioxolo [4, 5-h][2,3]benzodiazepine; LY300164 (see FIG. 1) is a selective, orally active noncompetitive antagonist of the AMPA (α-amino-3-hydroxy-5-methyl-4-isoxazolepropionic acid) subtype of glutamate excitatory amino acid receptors. Talampanel represents an orally active member of a class of noncompetitive antagonists of the AMPA subtype of glutamate excitatory amino acid receptors.

European Patent application EP0 492 485 discloses the compound 1-(4-aminophenyl)-3-acetyl-4-methyl-7,8-methylenedioxy-3,4-dihydro-5H-2,3-benzodiazepine. The compound is a potent and selective antagonist of the AMPA receptor and has been shown to be useful to treat a variety of neurological disorders. The (R) enantiomer of this compound, hereinafter referred to as (R)-7-acetyl-5-(4-aminophenyl)-8,9-dihydro-8-methyl-7H-1,3-dioxolo[4,5-h][2,3]benzodiazepine, is the most potent enantiomer (see, U.S. Pat. No. 6,288,057).

Talampanel has shown neuroprotective effects in vitro and in vivo. The neuroprotective efficacy of talampanel has been assessed in an in vitro model of AMPA-receptor-mediated excitotoxicity. Primary rat (ED18) hippocampal cultures (14-17 days in vitro) were treated overnight with an excitatory amino acid agonist in the presence or absence of drug. Neurodegeneration was assessed the next day by measurement of lactate dehydrogenase (LDH) released into the culture media. Talampanel afforded a dose-dependent neuroprotection against AMPA receptor mediated excitotoxicity with an IC₅₀ of 4 pM (May et al., Neurosci Lett. 262: 219-221 (1999)). Talampanel (2.5-5 mg/kg) also led to survival of hippocampal CA1 neurons after global ischemia in gerbils (Lodge et al., Neuropharmacology 35: 1681-1688 (1996)) and inhibited flexor reflexes in cats (Tarnawa et al, Eur. J. Pharmacol. 167: 193-199 (1989)).

Talampanel also showed anticonvulsant activity in a kindling model of epilepsy in rats. Talampanel suppressed chemically kindled seizures at 12.5 mg/kg and electrically kindled seizures at 20 mg/kg and it arrested seizures in a dose-related manner in a model of phenytoin-resistant status epilepticus (Borowicz et al., Eur. J. Pharmacol. 380: 67-72 (1999a); Borowicz et al., Pol. J. Pharmacol. 51: 103 (1999b)). Administrations of talampanel in patients with severe epilepsy not responsive to other drugs have shown positive results.

Processes for the synthesis of talampanel are provided in the literature see e.g., U.S. Pat. No. 6,288,057. An early original synthetic route involved large volumes of solvent and chromium oxide. An alternative synthesis uses a biocatalyst, the yeast Zygosaccharomyces rouxii, and a three-phase reaction system.

Pharmaceutical compositions of noncompetitive AMPA receptor antagonists can be prepared by methods well known in the art (see, U.S. Pat. No. 5,519,019 and 6,191,132). The compounds according to the invention are optionally formulated in a “pharmaceutically acceptable vehicle” with any of the well known pharmaceutically acceptable carriers, including diluents and excipients (see Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally “pharmaceutically acceptable carriers” are physiologically inert and non-toxic.

The compounds of the present invention may be provided in a pharmaceutically acceptable vehicle using formulation methods known to those of ordinary skill in the art. The compositions of the invention can be administered by standard routes. The compositions of the invention include those suitable for oral, inhalation, rectal, topical (transdermal) or parenteral (including subcutaneous, intramuscular, intravenous, intradermal, and intratracheal). In addition, polymers may be added according to standard methodologies in the art for sustained release of a given compound.

Pharmaceutical compositions comprising the compounds of all of the aspects of the present invention useful for treating pulmonary inflammation that are suitable for oral administration may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

The noncompetitive AMPA antagonist active compounds may be formulated for oral, buccal, intranasal, parenteral (e.g., intravenous, intramuscular or subcutaneous), transdermal (e.g., patch, ointment, cream or iontophoresis), or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycollate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). For buccal administration, the pharmaceutical composition may take the form of tablets or lozenges formulated in conventional manner.

The active compounds may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds may also be formulated in rectal compositions such as suppositories or retention enemas, e.g., containing conventional suppository bases such as cocoa butter or other glycerides.

For intranasal administration or administration by inhalation, the active compounds are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas.

For transdermal administration, the composition may take the form of patches, creams, ointments or iontophoresis formulated in conventional manner.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLES

### Example 1

### Effects on Post Traumatic Brain Injury

Rat Model. Fasted male Sprague-Dawley rats, weighing 290-390 g were employed. The animals were initially anesthetized with 3% halothane, 70% nitrous oxide, and a balance of oxygen. Rats were positioned in a stereotaxic frame following endotracheal intubation. A 4.8-mm craniotomy was made overlying the right parietal cortex (3.8 mm posterior to bregma and 2.5 mm lateral to midline) as previously described by Belayev et al., J. Neurotrauma, 16: 445-453 (1999) and Dietrich et al., J. Neuropathol. (Beri.) 87: 250-258 (1994). Next, a plastic injury tube was positioned over the exposed dura matter. Dental acrylic was then used to bond the injury tube to surrounding structures. After the acrylic had hardened, the injury tube was plugged with a Gelfoam sponge, the scalp closed with sutures, and the animal was returned to its cage and allowed to recover overnight.

Fluid-Percussion Brain Injury. The following day, the rats were reanesthetized with halothane, orotracheally intubated, and maintained on 70% nitrous oxide, 1.5% halothane, and a balance of oxygen. Temperature probes were inserted into the rectum (CMA/150 Temperature Controller, CMA/Microdialysis AB, Stockholm, Sweden) and the left temporalis muscle (Thermocouple Probe, Omega Engineering, Stanford, Conn.), and separate heating lamps were used to maintain rectal temperature and temporalis muscle temperature at 37.0-37.5° C. The right femoral artery and vein were catheterized for continuous blood pressure monitoring and periodic blood sampling for arterial gases, pH, hematocrit, and plasma glucose (measurements taken 15 min before TBI and 15 min after TBI). Rectal temperature and body weight was monitored before trauma and periodically for 7 days after trauma. Mean arterial blood pressure (MABP) was measured via an indwelling femoral arterial catheter connected to a precalibrated Statham pressure transducer (model P23XL, Viggo-Spectramed Inc., Oxnard, Calif., USA) and was recorded continuously (model RS3400 polygraph, Gould, Inc., Valley View, Ohio, USA). Serial measurements were made of arterial blood gases and pH (model ABL 330, Radiometer America, Inc. Westlake, Ohio, USA) and plasma glucose (model 2300 Stnt; Yellow Springs Instrument Co., Inc., Yellow Springs, Ohio, USA).

A fluid-percussion device was connected to the injury tube of the rat's skull. This device consisted of a saline-filled cylindrical Plexiglas reservoir having a rubber-coated piston at one end, a transducer housing to permit measurement of pressure transients, and a connecting device adapted to the rat's skull. Injury was induced by the descent of a pendulum, conveying a pressure transient of 1.5-2.0 atm. to the right dorsolateral parietal cortex (see Dietrich et al., J. Neuropathol. (Beri.), 87: 250-258 (1994)). Sham-operated animals (n=9) underwent similar surgical procedures but were not injured.

Drug-Infusion System. The femoral-vein cannula was tunneled subcutaneously so as to exit from the dorsal neck and was connected to a swivel device (Stoelting Co., Wood Dale, Ill., USA) attached to the roof of a spacious cage. This device allowed bi-directional rotation. PE-50 tubing extending from the other end of the device was joined to a disposable syringe attached to an infusion pump (KDS-100, KD Scientific, Boston, Mass., USA). This tethering system allowed the rats free movement without interfering with the patency of the cannula during drug administration.

Drug Administration. Talampanel (Lilly Research Laboratories, Indianapolis, Ind., USA; bolus infusion of 4 mg/kg followed by infusion of 4 mg/kg/h over 72 hr) or vehicle (40% 2-hydroxylpropyl-β-cyclodextrin solution, 0.3-ml bolus plus 0.3 ml/kg/hr over 72 hr) was administered I.V. beginning 30 min or 3 hr after trauma or sham operation.

The dosage was chosen because talampanel has been studied in vivo in a gerbil model of global ischemia (Lodge et al., Neuropharmacology, 35: 1681-1688 (1996)). In a preliminary experiment in which the talampanel was given by continuously infusion at doses of 5, 8 or 10 mg/kg, rats showed clinical signs of ataxia at doses of 8 and 10 mg/kg. Therefore, the dose was reduced to about 5 mg/kg. In a dose-response study of global ischemia in gerbils, Lodge et al. (1996) found that a 2.5-5 mg/kg-dose of talampanel was also significantly neuroprotective.

Histopathological Analysis of Brain Trauma. Animals were allowed to survive for 7 days following fluid-percussion brain injury. Brains were then perfusion-fixed with a mixture of 40% formaldehyde, glacial acetic acid, and methanol (FAM, 1:1:8 by volume), and brain blocks embedded in paraffin, according to the method of Nakayama et al., Neurology 38: 1667-1673 (1988). Ten micron-thick sections were cut in the coronal plane and stained with hematoxylin and eosin. Coronal sections at various levels (bregma levels: 0.7, −1.8, −3.3, −4.3, −5.8, −6.8, and −7.3 mm) were digitized by means of a Xillix CCD-based camera system with a Nikon macro lens and green filter, interfaced to an MCID image-analysis system (Imaging Research, St. Catherines, Ontario, Canada), from which data were exported to a DEC-Alpha workstation (Digital Equipment Corp., Maynard, Mass., USA) for processing. Two investigators blinded to the experimental groups then outlined the areas of contusion in the superficial, middle, and deep layers of parietal neocortex as well as the fimbria; these lesions were clearly demarcated on the sections.

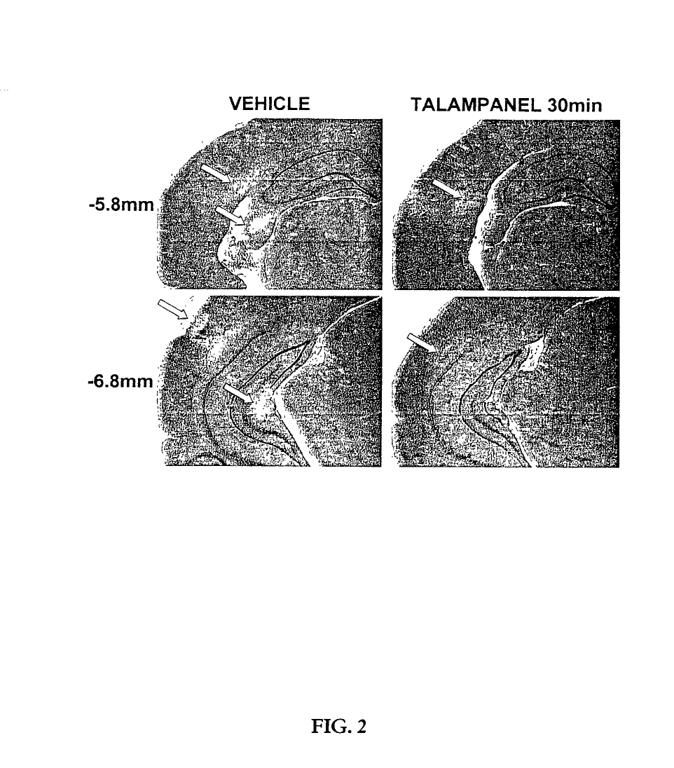

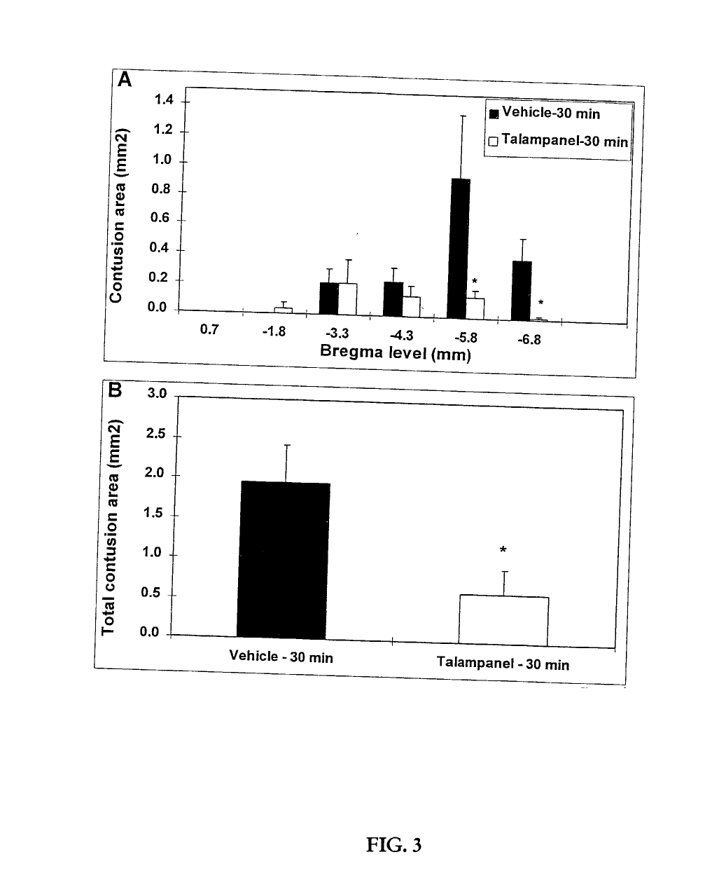

No pathologic changes were observed in any sham animals at any time points. Representative photomicrographs of vehicle-treated and talampanel-treated brains are shown in FIG. 2, and quantitative data are shown in FIG. 3 (treatment given at 30 min after trauma). In vehicle-treated rats, conspicuous multicentric foci of contusion were evident within the superficial, middle and deep layers of the dorsolateral parietal neocortex ipsilateral to TBI (FIG. 2). In addition, the subjacent fimbria exhibited consistent zones of contusion. By contrast, brains of talampanel-treated rats showed smaller contusion foci, typically confined to the deeper cortical layers (FIG. 2). Treatment with talampanel, when instituted 30 min after trauma, reduced mean contusion areas by 85% and 97%, respectively, at bregma levels −5.8 and −6.8 mm compared to vehicle-treated rats (FIG. 3A). Total contusion area was also reduced by 70% by treatment with talampanel at 30 min compared to vehicle-treated rats (1.79=0.42 vs. 0.54=0.25 mm²; see FIG. 3B).

When talampanel treatment was begun at 3 hr, the contusion area was reduced only at one bregma level (−6.8 mm) compared to vehicle-treated rats, but total contusion area did not differ statistically between talampanel (3-7 hr) and vehicle-treated rats (1.01=0.27 vs. 1.20=0.79 mm , respectively).

Histopathology of Hippocampus. In the hippocampus, numbers of normal pyramidal neurons were quantitated in the lateral, middle, and medial subsectors of the CA1 region, as well as in the CA3 sector bilaterally (bregma level, −4.3 mm) by an observer blinded to the experimental groups.

Quantitative analysis confirmed that treatment with talampanel starting at 30 min significantly attenuated ischemic damage in all three subsectors of the hippocampal CA1 sector compared to vehicle-treated rats (normal-neuron counts per ×40 microscopic field, right (ipsilateral) medial CA1: 80.3=2.0 [talampanel]vs. 66.3=2.1 [vehicle]; middle CA1: 71.5=2.0 vs. 60.3=2.2; lateral CA1: 74.5=3.0 vs. 63.0=3.2, respectively). By contrast, when talampanel treatment was begun at 3 hr, normal pyramidal-neuron counts were almost identical in both groups.

The numbers of normal neurons in the hippocampal CA3 sector averaged 28.8=2.5 in vehicle-treated rats, and 38.8=2.9 in the talampanel (30 min) group but did not differ statistically between groups. When talampanel treatment was begun at 3 hr, normal pyramidal-neuron counts in the hippocampal CA3 sector did not differ between groups.

The numbers of normal neurons in the hippocampal CA1 and CA3 sectors of sham animals, treated with vehicle or talampanel (30 min or 3 hr), were identical in all groups.

Ancillary Measurements from the Rat Model. Rectal and cranial (temporalis muscle) temperatures, arterial blood gases, plasma glucose arterial blood pressure, and body weight generally showed no significant differences between groups. There were no adverse behavioral side effects observed after talampanel administration to rats in this work. None of the animals died in this work.

Statistical Analysis. For each talampanel dose level, drug-treated and vehicle-treated groups were compared for total contusion areas, hippocampal neuron counts, and physiological variables by Student t tests. Inter-group differences were considered significant at the p<0.05 level. Values are presented as mean values±standard error of the mean (SEM).

Example 1 establishes that AMPA antagonist therapy instituted shortly after acute traumatic injury diminishes the size of cortical contusion and increased survival of pyramidal neurons of the CA1 sector of hippocampus. The protective effect of talampanel could not be explained by differences in body or brain temperature, arterial blood pressure or blood gases, because these variables were carefully controlled.

Talampanel was examined for potential cardiovascular effects in conscious male Sprague-Dawley rats for 2 hr, following single oral doses of 0.1, 0.3, 3 or 10 mg/kg. The cardiovascular variables measured included systolic, diastolic and mean arterial pressure, arterial pulse pressure, and heart rate. No changes were observed in these cardiovascular variables, suggesting that single therapeutic does of talampanel would not be expected to alter cardiovascular function. Thus, taken together, these in vitro and in vivo studies makes AMPA antagonists and specifically talampanel attractive candidates for a number of therapeutic indications (data not shown).

Without wishing to be bound to a particular mechanism of action, the inventors postulate that a possible explanation as to how blockade of the AMPA receptor attenuates injury at the cellular level (and thus how AMPA antagonists prevent CA1 neurosis after ischemia) is suggested by recent molecular studies involving the expression of AMPA receptor ionophore subunits in oocytes (Hollmann et al., Science 25:, 851-853 (1991)), which have shown that specific combinations of subunit molecules are permeable to calcium ions. Cells of the hippocampus allow Ca²⁺ entry through non-NMDA receptor-linked channels (see Iino et al., J. Physiol. (Lond.), 424: 151-165 (1990)). Specifically, the loss of the GluR2 subunit may be a necessary precondition to allow the AMPA receptor to conduct calcium ions (Hume et al., Science 253: 1028-1031 (1991)). Post-ischemic reductions in the mRNA for GluR2 compared with the expression of GluR1 and GluR3 in CA1, but not CA3, may afford an explanation for why AMPA antagonist surprisingly but effectively prevent calcium influx in the post-ischemic situation, thereby rescuing CA1 neurons (see Pellegrini-Giampietro et al., Proc. Natl. Acad. Sci. U.S.A., 89: 10499-10503 (1992)).

### Example 2

### Tableted Composition

Tablets or divided tablets containing talampanel as an active ingredient were prepared by standard tableting methods known in the art. An exemplary tableted composition was prepared by adding to 25 mg talampanel to inactive ingredients such as potato starch (43 mg); lactose (160 mg); polyvinylpyrrolidone (6 mg); magnesium stearate (1 mg) and talc (30 mg). Alternatively, tablets can be made by adding to 25 mg talampanel, inactive ingredients such as lactose (130 mg); maize starch (25 mg); microcrystalline cellulose (10 mg); gelatin (4 mg); talc (2 mg); stearin (1 mg); and magnesium stearate (1 mg).

### Equivalents

While the claimed invention has been described in detail and with reference to specific embodiments thereof, it will be apparent to one of ordinary skill in the art that various changes and modifications can be made to the claimed invention without departing from the spirit and scope thereof. Thus, for example those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A method for treating traumatic brain injury, comprising the step of administering to a subject in need thereof a therapeutically effective amount talampanel within a therapeutically effective time window.

2. The method of claim 1, wherein the step of talampanel is initiated less than 3 hours after brain injury.

3. The method of claim 4, wherein the step of talampanel is initiated about 30 minutes following brain injury.

4. The method of claim 1, wherein the therapeutically effective amount of talampanel is about 0.01-100 mg/kg per unit dose.

5. The method of claim 1, wherein the therapeutically effective amount of talampanel is administered in a divided dose.

6. The method of claim 1, wherein the therapeutically effective amount of talampanel is 2.5-5 mg/kg body weight.

7. The method of claim 1, wherein the therapeutically effective amount of talampanel is about 5 mg/kg body weight.

8. The method of claim 1, wherein the therapeutically effective amount of talampanel is about 4 mg/kg body weight/hr.

9. A pharmaceutical composition comprising a therapeutically effective amount of talampanel and a cyclodextrin carrier vehicle.

10. The pharmaceutical composition of claim 9, wherein the carrier is 2-hydroxylpropyl-β-cyclodextrin.
